(19) United States
(12) Patent Application Publication
(10) Pub. No.: US 2023/0325558 A1
(43) Pub. Date: Oct. 12, 2023

(54) DETECTOR OF ALIGNER LOW RETENTIVENESS AND ALIGNER FIT EVALUATION TOOL

(71) Applicant: Align Technology, Inc., San Jose, CA (US)

(72) Inventors: Arno KUKK, Moscow (RU); Daria BELYAEVA, Nizhny Novgorod (RU); Ekaterina KHARCHENKO, Vladivostok (RU)

(21) Appl. No.: 18/298,341

(22) Filed: Apr. 10, 2023

Related U.S. Application Data
(60) Provisional application No. 63/329,315, filed on Apr. 8, 2022.

(51) Int. Cl. G06F 30/20; A61C 7/00; A61C 13/34; A61C 7/08; G16H 20/40

(52) U.S. Cl. CPC G06F 30/20; A61C 7/002; A61C 13/34; A61C 7/08; G16H 20/40

(57) ABSTRACT

Methods and apparatuses for determining and improving the fit of orthodontic aligners. These methods and apparatuses may be used to identify regions of an aligner that may cause improper fitting. The methods and apparatuses may be used evaluate a dental treatment plan by predicting whether one or more aligners of the treatment plan will not be retained by the patient's dentition at various stages of the treatment plan.

### CROSS REFERENCE TO RELATED APPLICATIONS

This patent application claims priority to U.S. Provisional Patent Application No. 63/329,315, titled “DETECTOR OF ALIGNER LOW RETENTIVENESS AND ALIGNER FIT EVALUATION TOOL,” filed on Apr. 8, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods and apparatuses described herein are generally to the field of orthodontics, and more particularly to orthodontic aligners, and methods of evaluating the fit of the aligners to patients' teeth.

### BACKGROUND

Orthodontic and dental treatments using a series of patient-removable appliances (e.g., “aligners”) are useful for treating patients, and in particular for moving a patient's teeth to positions where function and/or aesthetics are optimized. Treatment planning is typically performed in conjunction with the dental professional (e.g., dentist, orthodontist, dental technician, etc.), by generating a model of the patient's teeth in a final configuration, and then breaking a treatment plan into a number of intermediate stages (steps) corresponding to individual appliances that are worn sequentially. This process may be interactive, adjusting the staging and in some cases the final target position, based on constraints on the movement of the teeth and the dental professional's preferences. Once the final treatment plan is finalized, the series of aligners may be manufactured corresponding to the treatment planning.

Aligners generally require flexibility for insertion and removal, but need rigidity to exert the force necessary for orthodontic tooth movement. Ideally, each align should fit snuggly against the teeth such that forces applied by an aligner causes prescribed movement of the teeth according to a treatment plan. If a shape or material property (e.g., rigidity or flexibility) of an aligner is off, the aligner may not properly fit on a patient's teeth. In some cases, an aligner may be difficult to place on the teeth, or may be difficult to remove from the teeth. In certain circumstances, an aligner may not stay in proper position on the patient's teeth. For example, a gap may form where the aligner doesn't fit flush against the patient's teeth. In more extreme cases, the aligner may pop partially off the patient's teeth. In some cases, an improper fitting aligner may cause patient discomfort, for example, if the aligner rubs against the patient's gums. Even though each aligner in a series has a prescribed shape, it can be difficult to predict whether any particular aligner will have fitting and/or retention problems.

The methods and apparatuses described herein may be used to improve detection of aligners that are at risk of improper fitting, as well as identifying and implementing adjustments to aligner designs to prevent improper fitting.

### SUMMARY

Described herein are methods and apparatuses (e.g., systems and devices) for predicting a retentiveness of a dental aligner (e.g., before it is made) and/or for detecting areas of a dental aligner that may cause the aligner to fit improperly (e.g., after it is made). The apparatus may include software analysis tools, which may be part of a dental treatment planning system for determining or modifying an orthodontic treatment plan for a patient's dental condition. The methods may be part of a method of generating and/or executing a dental treatment plan.

The methods and apparatuses described herein provide specific improvement over currently described systems in which the fit of one or more (and particularly a series) of dental appliances is determined only after fabrication of the appliance, or by trial-and-error. These methods and apparatuses may instead provide a dynamic method and apparatus (e.g., user interface) that may allow a user to quickly modify, in an iterative manner, specific subsets of features that may cause discrepancies and therefore poor fit. For example, a poor fit may result in a variety of different features (e.g., a mis-match of tooth shape, tooth position, and/or gingival line, a missing extracted tooth, or a present erupted tooth). By allowing the user to separately view and adjust these features in a user interface the overall design process may be vastly streamlined.

An aligner retentiveness predictor tool may be configured to predict whether dentitions at various stages of a treatment plan may result in producing one or more aligners having poor retention. This information may be used by a dental practitioner (e.g., dental treatment plan designer, dentist or orthodontist) to determine whether the treatment plan should be modified to improve the retentiveness of one or more aligners. The aligner retentiveness predictor tool may also provide recommendations as to locations for one or more dental attachments on the teeth to improve retention of an aligner.

A fit issue tool may be configured to identify what the root causes may be for a dental aligner that has been determined to not fit well. For example, the dental practitioner and/or the patient may complain about difficulty positioning the aligner onto the patient's teeth, too loose of an aligner, and/or discomfort from the aligner rubbing on soft tissues (e.g., gums). The fit issue tool may determine discrepancies between a first dentition model, which is used as a basis for forming the aligner, and a second dentition model, which represents the patient's a current dentition. The fit issue tool may categorize the discrepancies and determine which discrepancies are most likely to cause the improper fitting. The fit issue tool may also estimate a probability of each identified root cause for the improper fitting. The dental practitioner may use this information to decide whether to modify the treatment plan, and if so, determine the best ways to modify the treatment plan.

In some examples, a method for modifying a treatment plan based on one or more estimates of retentiveness of a dental aligner includes: receiving a three-dimensional (3D) model of a dentition in a stage of a treatment plan for which the dental aligner is configured to implement; identifying zones in the 3D model of the dentition that are predicted to provide low aligner retention; determining one or more attachment locations on the dentition estimated to increase the aligner retention; and providing a recommendation for the one or more attachment locations for use in combination with the dental aligner.

The 3D model of the dentition may correspond to the detention at initiation of the stage of the treatment plan.

The treatment plan may include multiple stages for moving teeth of the dentition toward a final position, wherein each of the multiple stages is associated with a corresponding aligner.

Estimating a retentiveness of a dental aligner may further include determining an aligner retentiveness of each of the multiple aligners, and providing a recommendation for one or more attachment locations on the dentition for use in combination with each of the multiple dental aligners.

Identifying zones in the 3D model of the dentition that are predicted to provide low aligner retention may include: dividing the 3D model into zones each having one or more teeth; calculating retention values for each of the zones, wherein the retention value of each zone is based on an average retention value for the one or more teeth in the corresponding zone.

Estimating a retentiveness of a dental aligner may further include determining a retention value for each tooth by: determining a reference vector parallel to a long axis of a tooth; determining surface normal vectors distributed across a surface of the tooth and normal to the surface of the tooth; calculating angles between the reference vector and each of the surface normal vectors; and calculating the retention value of the tooth based on a number of angles greater than 90 degrees.

In some examples, each surface normal vector may be associated with a polygon of a surface mesh of the tooth, and calculating the retention value may further include: calculating a cosine of each of the angles between the reference vector and corresponding surface normal vector; identifying polygons having surface normal vectors with cosines less than zero; measuring areas of each of the identified polygons; and adding the areas of the identified polygons together.

Estimating a retentiveness of a dental aligner may further include determining a shape and size of one or more retention-enhancing attachments at the recommended one or more attachment locations.

In some examples, estimating a retentiveness of a dental aligner may further include: adding one or more retention-enhancing attachments at the recommended one or more attachment locations to the stage of the treatment plan; and modifying the treatment plan based on the addition of the one or more retention-enhancing attachments.

Estimating a retentiveness of a dental aligner may further include fabricating one or more dental aligners based on the modified treatment plan.

In some examples, a non-transient, computer-readable medium may contain program instructions for modifying a treatment plan based on one or more estimates of retentiveness of a dental aligner, the program instructions causing a processor to: receive a three-dimensional (3D) model of a dentition in a stage of a treatment plan for which the dental aligner is configured to implement; identify zones in the 3D model of the dentition that are predicted to provide low aligner retention; determine one or more attachment locations on the dentition estimated to increase the aligner retention; and provide a recommendation for the one or more attachment locations for use in combination with the dental aligner.

Identifying zones in the 3D model of the dentition that are predicted to provide low aligner retention may include: dividing the 3D model into zones each having one or more teeth; and calculating retention values for each of the zones, wherein the retention value of each zone is based on an average retention value for the one or more teeth in the corresponding zone.

Program instructions of the non-transient, computer-readable medium may include determining a retention value for each tooth by: determining a reference vector parallel to a long axis of a tooth; determining surface normal vectors distributed across a surface of the tooth and normal to the surface of the tooth; calculating angles between the reference vector and each of the surface normal vectors; and calculating the retention value of the tooth based on a number of angles greater than 90 degrees.

Each surface normal vector may be associated with a polygon of a surface mesh of the tooth, where calculating the retention value may further include: calculating a cosine of each of the angles between the reference vector and corresponding surface normal vector; identifying polygons having surface normal vectors with cosines less than zero; measuring areas of each of the identified polygons; and adding the areas of the identified polygons together.

In some examples, a method for displaying a root cause of an improperly fitting dental aligner includes: determining discrepancies between a first dentition model and a second dentition model, wherein the first dentition model is a basis for forming the dental aligner, and wherein the second dentition model is based on a current configuration of a patient's dentition; categorizing the discrepancies according to a discrepancy type, wherein the discrepancy type includes a tooth shape, a tooth position, a gingival line, an extracted tooth, or an erupted tooth; interactively displaying a comparison dentition model highlighting one or more of the discrepancies, wherein a user control allows a user to choose to display the one or more discrepancies based on the discrepancy type; calculating a probability of one or more root causes of an improper fit of the dental aligner, wherein the probability is based on identifying one or more of the discrepancies having a high risk of contributing to the improper fit of the dental aligner; and displaying the probability of one or more root causes.

Identifying one or more of the discrepancies having a high risk of causing an improper fit may be based on threshold discrepancy values.

Identifying one or more of the discrepancies having a high risk of causing an improper fit may be based on a type of the discrepancy, a location of the discrepancy, or a type and location of the discrepancy.

The first and second dentition models may be three-dimensional (3D) models. The comparison dentition model may be three-dimensional (3D) model.

The user control may include one or more of: a radio button, a slide bar, a switch, and drop-down menu.

Determining a root cause of an improperly fitting dental aligner may further include: modifying a dental plan for the patent; and fabricating one or more dental aligners based on the modified treatment plan.

In some examples, a non-transient, computer-readable medium may contain program instructions for displaying a root cause of an improperly fitting dental aligner, the program instructions causing a processor to: determine discrepancies between a first dentition model and a second dentition model, wherein the first dentition model is a basis for forming the dental aligner, and wherein the second dentition model is based on a current configuration of a patient's dentition; categorize the discrepancies according to a discrepancy type, wherein the discrepancy type includes a tooth shape, a tooth position, a gingival line, an extracted tooth, or an erupted tooth; interactively display a comparison dentition model highlighting one or more of the discrepancies, wherein a user control allows a user to choose to display the one or more discrepancies based on the discrepancy type; calculate a probability of one or more root causes of an improper fit of the dental aligner, wherein the probability is based on identifying one or more of the discrepancies having a high risk of contributing to the improper fit of the dental aligner; and display the probability of one or more root causes.

For example, described herein are methods for digital simulation of a retentiveness of a dental aligner, the method comprising: initializing a 3D model of a patient's dentition based on a stage of a treatment plan corresponding to the dental aligner; generating a plurality of zones, each having one or more teeth from the 3D model of the patient's dentition; simulating a retention value for each of the zones, based on one or more angles between one or more surface normal vectors of one or more teeth within each zone, and one or more reference vectors parallel to a long axis of each of the one or more teeth within each zone; and outputting one or more attachment locations on the patient's dentition based on the retention values.

Any of these methods may output an indicator of the probability that dental aligner will improperly fit the patient, instead of (or in addition to) outputting attachment locations to that may address these retention/fit issues. For example, described herein are methods for digital simulation of a retentiveness of a dental aligner (and software configured to perform these methods) comprising: initializing a 3D model of a patient's dentition based on a stage of a treatment plan corresponding to the dental aligner; generating a plurality of zones, each having one or more teeth from the 3D model of the patient's dentition; simulating a retention value for each of the zones, based on one or more angles between one or more surface normal vectors of one or more teeth within each zone, and one or more reference vectors parallel to a long axis of each of the one or more teeth within each zone; and outputting one or more indicators of the probability that the dental aligner will improperly fit the patient's dentition based on the retention values.

In any of these examples the 3D model of the dentition may correspond to the detention at initiation of the stage of the treatment plan. The treatment plan may include multiple stages for moving teeth of the dentition toward a final position, wherein each of the multiple stages is associated with a corresponding aligner.

Any of these methods may include repeating the steps of: initializing, generating, simulation and outputting for each of a plurality of aligners of the different stages of the treatment plan. In any of these methods generating the plurality of zones may include: dividing the 3D model into zones each having one or more teeth, wherein the retention value of each zone is based on an average retention value for the one or more teeth in the corresponding zone.

Simulating the retention value may include: determining a reference vector parallel to a long axis of a tooth; determining the surface normal vectors distributed across a surface of the tooth and normal to the surface of the tooth; and calculating angles between the reference vector and each of the surface normal vectors. Simulating the retention value of the zones may be based on a number of angles greater than 90 degrees.

Any of these methods may include determining a shape and size of one or more retention-enhancing attachments at the one or more attachment locations. In some examples the methods include: adding one or more retention-enhancing attachments at the one or more attachment locations to the stage of the treatment plan; and modifying the treatment plan based on the addition of the one or more retention-enhancing attachments. Any of these methods may include fabricating one or more dental aligners based on the modified treatment plan.

As mentioned above, also described herein are systems for performing any of these methods and non-transient, computer-readable medium containing program instructions for performing any of these methods. The systems may include a memory and one or more processors.

For example, a non-transient, computer-readable medium containing program instructions for modifying a treatment plan based on one or more estimates of retentiveness of a dental aligner may be configured so that the program instructions cause a processor to: initialize a 3D model of a patient's dentition based on the stage of a treatment plan corresponding to the dental aligner; generate a plurality of zones, each having one or more teeth from the 3D model of the patient's dentition; simulate a retention value for each of the zones, based on one or more angles between one or more surface normal vectors of one or more teeth within each zone, and one or more reference vectors parallel to a long axis of each of the one or more teeth within each zone; and output one or more attachment locations on the patient's dentition based on the retention values.

Also described herein are methods for enhancing an orthodontic aligner design graphical user interface (GUI), the method comprising: simulating a first digital model of a patient's dentition based on a dental aligner of a treatment plan; simulating a second digital model of the patient's dentition based on a configuration of the patient's dentition; receiving via the GIU, a user selection of one or more discrepancy type between the first digital model and the second digital model, wherein the discrepancy type comprises: a tooth shape, a tooth position, a gingival line, an extracted tooth, or an erupted tooth; displaying a comparison dentition model highlighting one or more discrepancies between the first digital model and the second digital model, based on the one or more selected discrepancy type; and modifying the display to indicate a probability that dental aligner will improperly fit the patient's dentition.

Any of these methods may include identifying the one or more discrepancies based on one or more threshold discrepancy values. Identifying the one or more of the discrepancies may be further based on the discrepancy type, a location of the discrepancy, or a type and location of the discrepancy. These methods may include forming the comparison dentition model as an overlay of the first digital model and the second digital model.

Displaying may include dynamically labeling regions of the comparison dentition model corresponding to the user selected one or more discrepancy types with an indicator of the probability that the dental aligner will properly or improperly fit the patient's dentition. The indicator may be a numeric value (e.g., percent, scaled value, etc.), a qualitative indicator (“high,” “moderate,” “low”) or a graphical (e.g., red, yellow, green) indicator. The display may provide an interpretation of the indicator as more or less likely that the aligner will fit.

Any of these methods may include modifying the treatment plan based on user input after modifying the display to indicate the probability that the dental aligner will improperly fit; and fabricating one or more dental aligners based on the modified treatment plan.

Also described herein are non-transient, computer-readable medium containing program instructions for displaying a root cause of an improperly fitting dental aligner, the program instructions causing a processor to: simulate a first digital model of a patient's dentition based on a dental aligner of a treatment plan; simulate a second digital model of the patient's dentition based on a configuration of the patient's dentition; receive via the GIU, a user selection of one or more discrepancy type between the first digital model and the second digital model, wherein the discrepancy type comprises: a tooth shape, a tooth position, a gingival line, an extracted tooth, or an erupted tooth; display a comparison dentition model highlighting one or more discrepancies between the first digital model and the second digital model, based on the one or more selected discrepancy type; and modify the display to indicate a probability that dental aligner will improperly fit the patient's dentition.

All of the methods and apparatuses described herein, in any combination, are herein contemplated and can be used to achieve the benefits as described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 is a flowchart indicating an example method for determining a retentiveness of a dental aligner.

FIG. 2A illustrates a side view of tooth showing an example of how a shape and orientation of a tooth can be used to determine a retention value or score of the tooth.

FIG. 2B illustrates an example user interface showing calculated retention values for different zones of a dentition.

FIG. 3 is a diagram showing an example data structure for an aligner retentiveness predictor tool.

FIG. 4 is a flowchart indicating an example method for determining a root cause of an improperly fitting dental aligner.

FIG. 5 illustrates an example dentition model used as a basis to form a dental aligner.

FIG. 6 illustrates an example comparison dentition model that shows differences between a dentition model used as a basis to form a dental aligner (original dentition model) and a dentition model representing a patient's current dentition (current dentition model).

FIG. 7 illustrates another example comparison dentition model showing discrepancies of the incisor teeth.

FIG. 8 illustrates an example user interface display window showing a brief summary of shape discrepancy metrics between an original dentition model and a current dentition model.

FIG. 9 illustrates an example a comparison dentition model displayed using tooth shifts in the individual teeth.

FIG. 10 illustrates an example of a comparison dentition model showing differences in gingival lines associated with a particular tooth.

FIG. 11 illustrates an example of a comparison dentition model showing the location of an unextracted tooth and an unextracted pontic tooth.

FIG. 12A illustrates an example of a user interface toolbar for controlling viewing aspects of one or more dentition models.

FIG. 12B illustrates a close-up view of an example drop-down menu for display scenarios in the toolbar of FIG. 12A.

FIG. 13 illustrates an example dashboard view of a Fit Issue Tool that summarizes statistical findings of a fit issue analysis.

FIG. 14 is a diagram showing an example data structure for fit issue tool.

### DETAILED DESCRIPTION

The methods and systems described herein may relate to series of dental appliances (e.g., “aligners”) for repositioning teeth from an initial tooth arrangement to a final tooth arrangement. Repositioning of the teeth is implemented in accordance with a prescribed treatment plan for moving the teeth from the initial arrangement toward the final arrangement over a period of time. The treatment plan is usually partitioned into multiple incremental intermediate stages, typically with one aligner associated with each stage. For example, a treatment plan that includes 25 stages may involve the use of 25 aligners. An aligner associated with a particular stage of the treatment plan is configured to move the teeth from a first arrangement at the beginning of the particular stage toward a second arrangement at the end of the particular stage.

The treatment plan may be represented by one or more digital three-dimensional (3D) models of the patient's dentition. Typically, an initial 3D model of the patient's dentition is obtained by scanning the patient's dentition using a dental scanner. A final 3D model representing a desired configuration of the patient's teeth may be determined based on the initial 3D model. Each stage of the treatment plan may be associated with a first intermediate 3D model representing the dentition at the beginning of the treatment stage and second intermediate 3D model representing the dentition at the end of the treatment stage.

Each aligner is configured to fit on the patient dentition, and is typically configured to be removable by the patient. The individual aligners may be made of a polymeric shell and shaped to have a teeth-receiving cavity for receiving the teeth of a dental arch. The teeth-receiving cavity may have a geometry corresponding to an end tooth arrangement intended for that stage of the treatment plan. That is, when an aligner is first worn by the subject, certain teeth will be misaligned relative to an undeformed geometry of the aligner cavity. The aligner, however, is sufficiently resilient to accommodate or conform to the misaligned teeth, and will apply sufficient resilient force against such misaligned teeth to reposition the teeth toward the end arrangement desired for that treatment stage.

In some cases, an aligner may be configured to apply the repositioning forces on the teeth with one or more dental attachments that are temporarily bonded to the patient's teeth. Attachments may have different shapes and sizes and be made different materials, depending on their function. In many cases, attachments are small tooth-colored ridges made of orthodontic material designed to blend with the tooth enamel. The aligner may be configured to provide directed pressure against the attachments, making more complex directional tooth movement possible. The aligner may have indentations with shapes matching those of the corresponding attachments such that the aligner can fit snugly and smoothly over them.

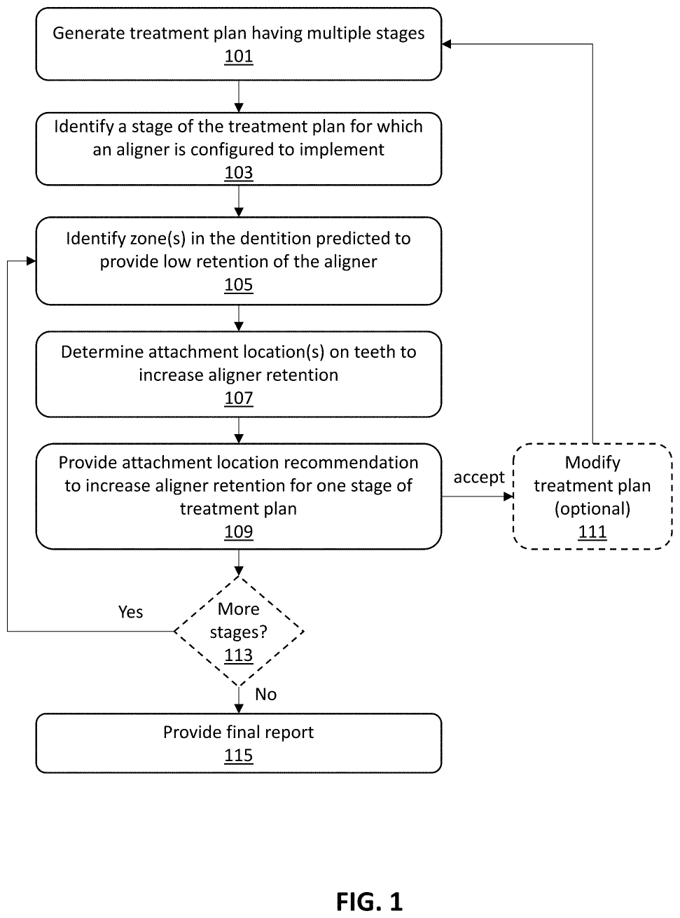

In some circumstances, a patient's dental configuration may make it difficult to make a proper fitting aligner. For example, the patient may be missing one or more teeth (e.g., deciduous teeth), thereby providing less traction for the aligner along spaces where the teeth used to be. In some instances, certain teeth may be small or otherwise have shapes that make an aligner easy to slip or pop off the patient's dentition. FIGS. 1-3 illustrate aspects of an aligner retentiveness analysis tool used for estimating a retentiveness of an aligner based on the dentition at a stage of a dental treatment plan, and for providing recommendations for improving the retentiveness of the aligner. In some cases, the analysis is based on a 3D model of the patient's dentition during a stage of the treatment plan (e.g., not based on the aligner). Results from the analysis may be used (e.g., by a dental professional) to determine whether to adjust one or more stages of the treatment plan.

FIG. 1 is a high-level flowchart illustrating an example method of estimating a retentiveness of a dental aligner. At 101, a treatment plan having multiple stages is generated. The treatment plan may be represented by 3D models of the patient's dentition. For example, an initial or pre-treatment 3D model may represent the patient's dentition before treatment, a final 3D model may represent a target dentition after treatment is complete, and intermediate 3D models may represent the patent's dentition at intermediate stages from the initial position toward the final target position. In some cases, the initial or pre-treatment 3D model may correspond to, or be derived from, one or more 3D scans of the patient's dentition.

At 103, one stage of the treatment plan is identified for analysis. The stage of the treatment plan may be chosen based on the likelihood of retention issues. For example, if the patient recently lost a tooth (e.g., a deciduous tooth), the next stage in the treatment plan may be chosen. In some cases, a late stage in the treatment plan may be chosen since aligner retention issues may be more likely when the teeth are in more aligned orientations/positions. Once a stage of the treatment plan is chosen, a 3D model associated with the treatment stage is accessed. The 3D model may correspond to the dentition at initiation of that treatment stage. This is because the aligner used to implement a treatment stage is configured to apply forces to the dentition at initiation of the stage toward a desired/final dentition configuration at the end of the treatment stage.

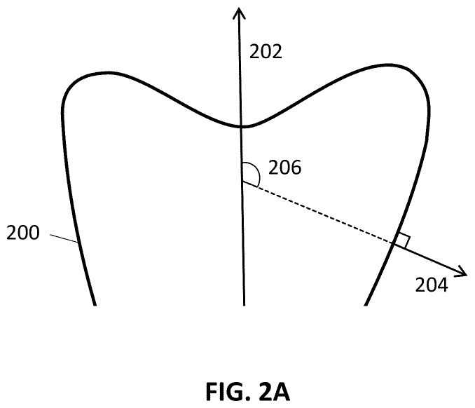

Once a stage of the treatment plan is chosen, at 105, one or more zones in the 3D model identified as predicted to provide low retention of an aligner are identified. In many cases, such low retentiveness zone(s) may include one or more posterior teeth. In some cases, the low retentiveness zone(s) can include some or all posterior teeth. In some cases, the low retentiveness zone(s) may be restricted one or more terminal posterior teeth. Identifying low retentiveness zones can include estimating retention force based on teeth shapes, teeth inclination and/or arch form. Details of such analyses are described in detail herein with reference to FIGS. 2-4.

If one or more low retentiveness zones are detected, at 107, attachment location(s) on the teeth to increase aligner retention may be determined. Such retention-enhancing attachment(s) can provide points of friction for the aligner to grip onto, thereby providing a retention force to retain the aligner to the teeth. The retention-enhancing attachment(s) may be in addition to any attachments originally included in the treatment plan stage, or may be the only attachments in the treatment plan stage. In some cases, the retention-enhancing attachment(s) may replace one or more attachments originally included in the treatment plan stage. The shape and size of the retention-enhancing attachment(s) may depend on their location on the teeth/tooth and a desired direction of retention force(s).

Once the retention-enhancing attachment(s) are determined, at 109, a recommendation is provided, for example, via a user interface of an electronic device (e.g., computer, tablet or phone). A dental professional (e.g., dentist, orthodontist or dental appliance designer) can decide whether to include the recommended retention-enhancing attachment(s) in the treatment plan stage. If the dental professional decides to include the recommended retention-enhancing attachment(s), the attachments may be incorporated into the dental plan 111. In some cases, the changes are entered into an orthodontic treatment plan software (e.g., via plugin) to determine a modified treatment plan based on the added attachment(s). Optionally, the user may evaluate the retentiveness of one or more dental aligners of the modified treatment plan 101. This process may be repeated as needed or desired by the user.

At 113, a determination is optionally made as to whether there are any more stages of the treatment plan to evaluate. If there are one or more additional stages, one of those stages may optionally be evaluated to determine whether there are any zones predicted to provide low retention of an associated aligner of that stage 105, determine attachment location(s) to increase aligner retention 107, and provide attachment location recommendations 109. Such analysis may be repeated, for example, for all the stages of the treatment plan. This analysis may be performed automatically or as chosen by the user. For example, a user interface may allow a user to choose whether to evaluate one or more stages of the treatment plan. If there are no more treatment plan stages to evaluate, or the user chooses not to evaluate any more treatment plan stages, a final report may be provided 115. The final report may include a summary of aligner retention prediction results for each of the evaluated treatment plan stages. The dental professional can choose whether to implement any of the recommendations in one or more stages of the treatment plan. If implemented, one or more aligners of the treatment plan can be manufactured according to the modified treatment plan.

The retentiveness of an aligner may be estimated based on the shape and orientation of one or more teeth in the patient's dentition. In any of these methods and apparatuses, the retentiveness may be estimated based on the angle of the side (e.g., the buccal and/or lingual side) of all or some of the teeth relative to the long axis of the tooth (e.g., an axis extending normal to the occlusal surface of the tooth). FIG. 2A shows a side view of a tooth 200 to illustrate how a shape and orientation of the tooth 200 can be used to determine a retention value or score of the tooth 200. The estimation may be based on a 3D model of the tooth 200 in predetermined orientation. For example, the tooth 200 may be oriented in accordance with a position/orientation of the tooth at initiation of a particular stage of the treatment plan. A reference vector 202 that is parallel to a long axis of the tooth 200 is determined. In addition, surface normal vectors, e.g., 204, distributed across a surface of the tooth 200 and that are normal to the surface of the tooth 200 are determined. For example, each surface normal vector, e.g., 204, may be associated with a polygon of a surface mesh of the 3D model. Angles, e.g., 206, between the reference vector 202 and each of the surface normal vectors, e.g., 204, are then calculated. These angles, e.g., 206, are associated with a degree in which portions of the tooth surface can provide a retention force for an aligner. For example, surface portions associated with angles, e.g., 206, greater than 90 degrees can contribute a positive retention force for an aligner. Surface portions associated with angles greater than 90 degrees, such as the surface associated with surface normal vector 204 in FIG. 2, may be referred to as an undercut surface portion. A retention value or score of the tooth 200 may be estimated based on the number of angles, e.g., 206, that are greater than 90 degrees. In some cases, this calculation includes calculating a cosine of each of the angle(s), e.g., 206, based on vectors that are normal 204 to the surface of identified polygons. The angles of each of the identified polygons may be measured and added together to provide a weighting based on surface area. The retention value/score of the tooth 200 may be based on this sum. In some cases, the retention value/score may be based on a range. For example, the tooth 200 may be estimated to have a low retention value/score if the area of retention surfaces is above a first threshold, a medium retention value/score if the area of retention surfaces is above a second threshold greater than the first threshold, and a high retention value/score if the area of retention surfaces is above a third threshold greater than the second threshold. Retention scores may be estimated for individual teeth and/or for groups of the teeth (or all of the teeth).

In some examples, retention values/scores for various zones of a dentition may be calculated based on the retention values/scores of the teeth. FIG. 2B illustrates an example dentition showing retention scores for different zones of the dentition (e.g., as shown in a user interface). In this example, retention values/scores are provided for three zones: Zone 1, Zone 2, and Zone 3. Zone 1 includes the lower left 8 tooth (LL8), the lower left 7 tooth (LL7), and the lower left 6 tooth (LL6), which correspond to the three last posterior teeth of the left side of the lower dentition. Zone 2 includes all the teeth of Zone 1 and also the lower left 5 tooth (LL5), which correspond to the four last posterior teeth of the left side of the lower dentition. Zone 3 includes all the teeth of Zone 2 and also the lower left 4 tooth (LL4), which correspond to the five last posterior teeth of the left side of the lower dentition. In this case, the retention values of each of the Zones 1-3 corresponds to an average retention value of the teeth of each zone. For example, Zone 1 has a retention value/score of R3, Zone 2 has a retention value/score of R4, and Zone 3 has a retention value/score of R5. A predicted lack of retention of an aligner can be determined based on a comparing retention values/scores of the zones to threshold values. In this example, a lack of retentive forces in the lower left quadrant of the dentition may be detected if R3 of Zone 1 is less than a first threshold value T3, R4 of Zone 2 is less than a second threshold value T4, or R5 of Zone 3 is less than a third threshold value T5. Similar calculations may be performed for other regions of the dentition (e.g., lower right quadrant). These calculations may then be used to determine a retentiveness of the aligner. For example, if it is determined that a left or right quadrant of a dentition lacks retentive forces, the user may be informed that the aligner is predicted to lack retentiveness. If a lack of retentiveness is determined, one or more retention-enhancing attachment locations for increasing the retention forces may be determined. For example, if one of Zones 1-3 has a low retention value/score, one or more attachments may be recommended on one or more teeth within Zones 1-3. In some cases, the recommendation is based on the retention value/score of a particular tooth. For example, if tooth LL7 has a low retention value (e.g., lower than a threshold value), an attachment may be recommended for the tooth LL7. In some examples, a shape, size and/or specific location on a tooth of one or more retention-enhancing attachments is recommended.

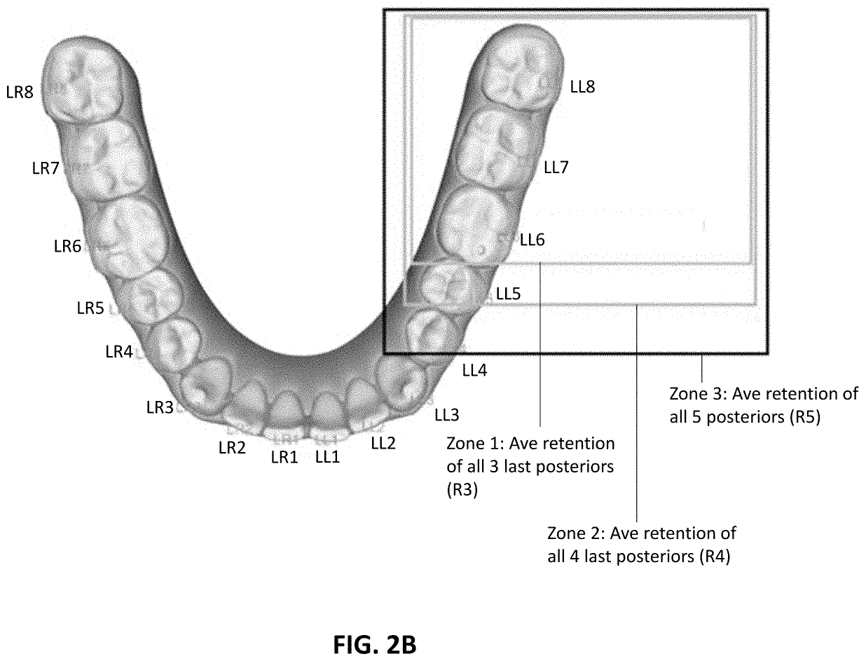

FIG. 3 is a diagram showing an example of an aligner retentiveness predictor tool 300. The aligner retentiveness predictor tool 300 may be incorporated into a portion of another system (e.g., a general treatment planning system) and may therefore also be referred to as a sub-system. In some cases, the aligner retentiveness predictor tool 300 may be an add-on (e.g., plug-in) to the general treatment planning system. In any of the method and apparatuses described herein the aligner retentiveness predictor tool 300 may be invoked by a user control, such as a tab, button, etc., as part of treatment planning system, or may be separately invoked. In FIG. 3, the aligner retentiveness predictor tool 300 may include a plurality of engines and datastores. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. The aligner retentiveness predictor tool 300 may include or be part of a computer-readable medium, and may include an input engine 314 (e.g., providing and/or allowing access to the patient's dentition data and/or 3D dentition models related to a dental treatment plan). All or some of the dentition data may be stored in a datastore 316.

In any of these methods and apparatuses (e.g., systems), a computer system can be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described herein can be cloud-based datastores. A cloud-based datastore is a datastore that is compatible with cloud-based computing systems and engines.

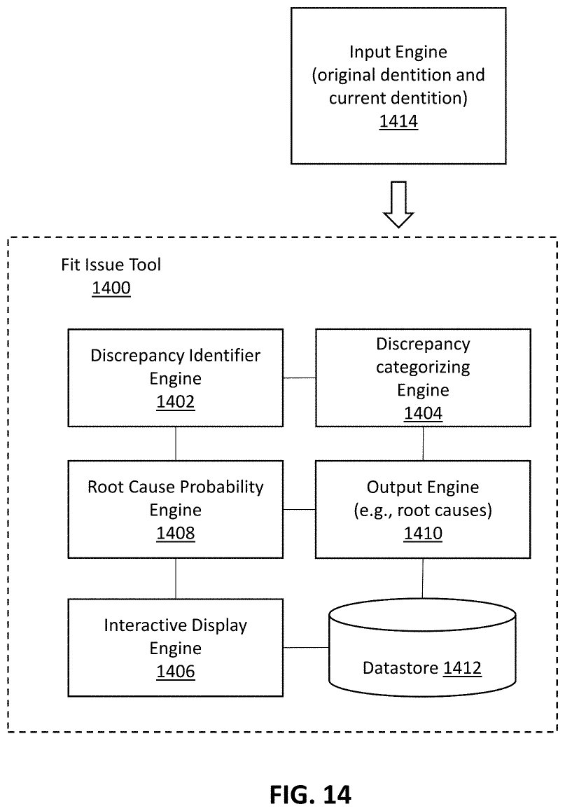

The fit issue tool 1400 may include a computer-readable medium. The modules/engines may be coupled to one another (e.g., example couplings are shown in FIG. 14 by the interconnecting lines) or to modules/engines not explicitly shown in FIG. 14. The computer-readable medium may include any computer-readable medium, including without limitation a bus, a wired network, a wireless network, or some combination thereof. The engine described herein may implement one or more automated agents, including machine learning agents.

The aligner retentiveness predictor tool 300 may include a tooth retention calculator engine 302 that may calculate a retention value of one or more teeth of the dentition. This may include, for each tooth, determining a reference vector parallel to a long axis of the tooth, determining surface normal vectors distributed across a surface of the tooth, calculating angles between the reference vector and each of the surface normal vectors, and adding areas of the surface mesh that correspond to high retention angles. The aligner retentiveness predictor tool 300 may also include a zone divider engine 304 that may divide the dentition into zones each having one or more teeth. In some examples, the dentition is divided into quadrants, with each quadrant including the posterior teeth of the dentition. The aligner retentiveness predictor tool 300 may also include a zone retention calculator engine 306 that calculates retention values for each zone. The aligner retentiveness predictor tool 300 may also include an attachment recommendation engine 310, which may determine locations on one or more teeth (e.g., in zone(s) determined to have low retention values) for increasing the retention values. The aligner retentiveness predictor tool 300 may also include an interactive display engine 312 that displays an interactive user interface. The user interface may display the one or more 3D models of a dentition indicating teeth and/or zones that have low retention values. In some examples, the user interface may display retention values for all teeth and/or zones. The user interface may also display recommended attachment locations on the 3D model of the dentition. An output engine 303 may output results from the root cause analysis, for example, in the user interface.

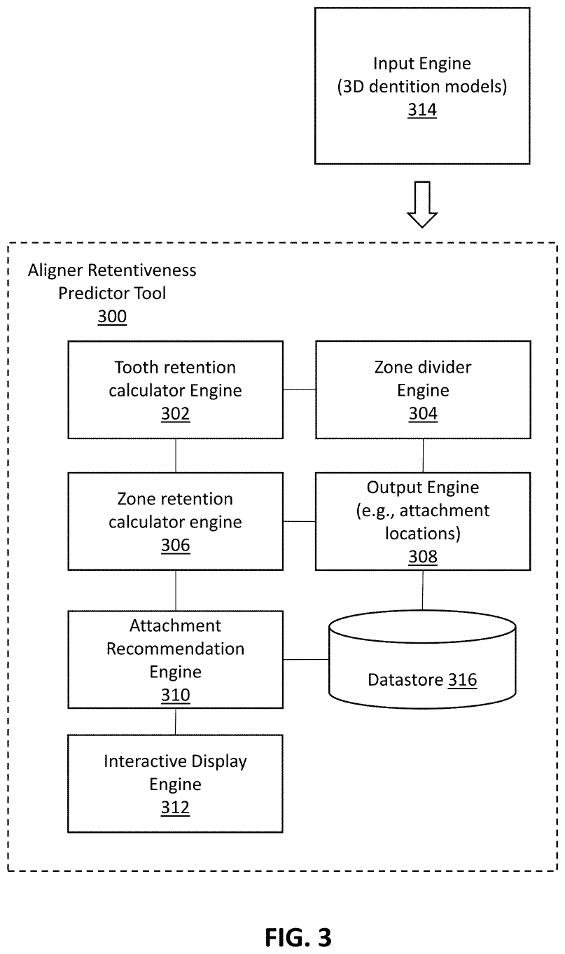

In some situations, a patient or dental professional may find that an aligner that has already been made does not fit properly. For example, the aligner may be too difficult to place on the teeth or remove from the teeth, or the aligner may tend to pop off the teeth. In some situations, the aligner may cause discomfort by rubbing against the patient's soft tissues, such as the gums or palate. FIGS. 4-14 illustrate aspects of a “Fit Issue Tool” for determining a root cause of an improperly fitting aligner.

FIG. 4 is a high-level flowchart illustrating an example method of determining a root cause of an improperly fitting dental aligner. At 401, a first dentition model, which was used as a basis to manufacture the aligner, is compared to a second dentition model based on a new scan of the patient's dentition. The first dentition model represents the patient's dentition as some point prior to the manufacturer of one or more aligners of a dental treatment plan. As such, aspects of the patient's dentition may have changed. For example, the patient may have one or more new missing teeth (e.g., deciduous teeth or otherwise extracted teeth), one or more newly erupted teeth, the position of the patient's teeth may have shifted, or the gingival line location may have changed. In some cases, the first dental model may be based on an intermediate model of a sequence of models derived from a treatment plan, and which may have inaccurate dimensions compared to the patient's actual dentition. A new scan, which may be taken in the dental professional's office, provides a second dental model that is up to date as to the patient's current dental condition. The first and second dentition models may be digital 3D models. The first and second dentition models may be in the same digital formal (e.g., file type), or converted in the same digital format.

At 403, discrepancies between the first and second dentition models are determined. For example, positional data of the second dentition model can be compared to positional data the first dentition model to determine any positional discrepancies. The discrepancies may be, for example, differences in a shape of one or more teeth, a position of one or more teeth, differences in a position of a gingival line one or more teeth, one or more missing teeth, and one or more new teeth (e.g., tooth eruption or pontic tooth).

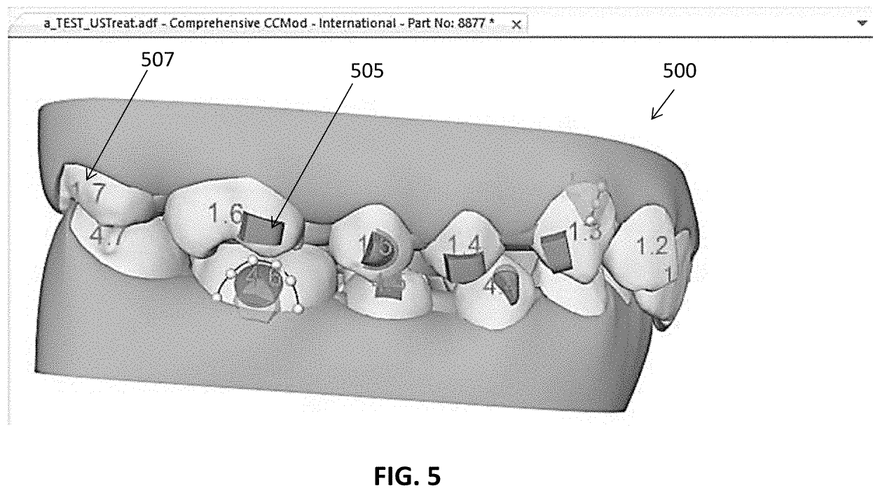

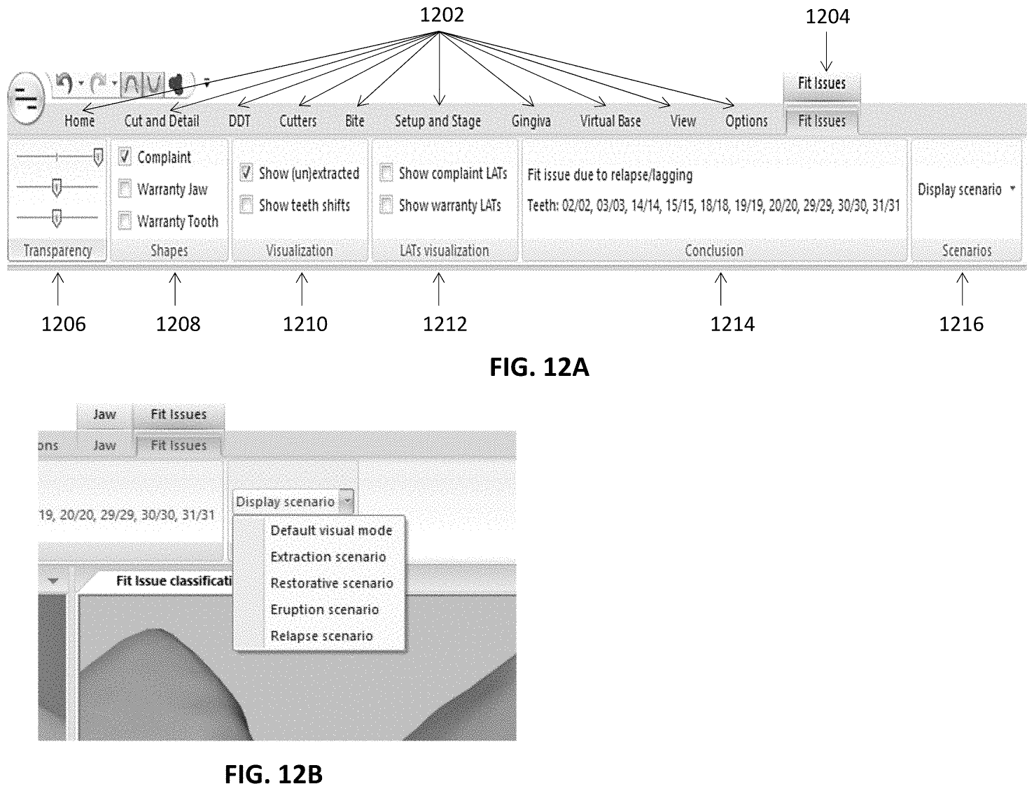

At 405, the discrepancies are categorized according to discrepancy type. Discrepancy type may include, for example, tooth shape, tooth position, gingival line position, missing tooth and new tooth. At 407, a comparison dentition model, which includes the discrepancies between the first and second dentition models, is displayed on a user interface. See, e.g., FIGS. 6, 7, 9, 10 and 11. The user interface may be interactive. For example, the user interface may include one or more controls (e.g., radio buttons, slide bars, switches, and drop-down menus) that allow a user to choose whether to display (e.g., highlight) one or more of the discrepancies, and which of the discrepancies to display (e.g., highlight), based on the discrepancy types. See, e.g., FIGS. 12A and 12B.

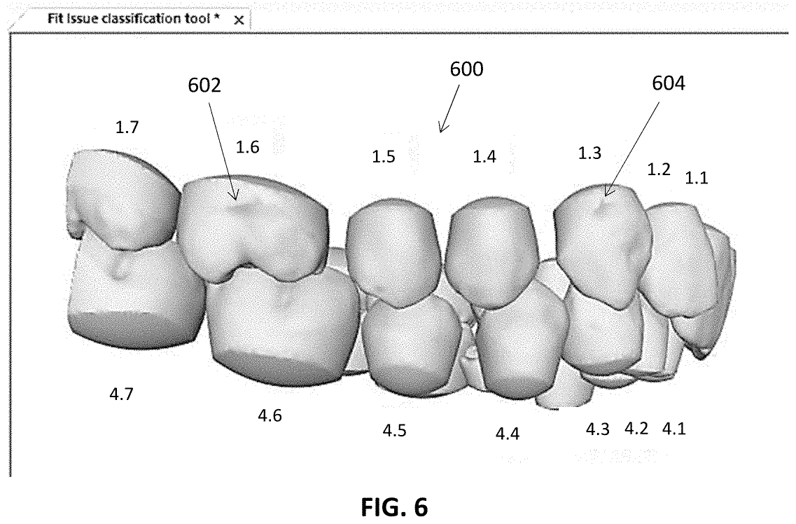

At 409, a probability of one or more root causes of the improper fit of the aligner is calculated based on the discrepancies. Determining the probably of the root cause(s) may involve associating each of the categorized discrepancies with a root cause. For example, differences in tooth positions/shapes and/or gingival lines may be associated with root cause of relapse or lagging of tooth movement toward a desired dentition (according to a treatment plan). Relapse/lagging may be due to a patient's lack of compliance (e.g., not wearing aligners as prescribed), or may be due to a health issue affecting the patient's dentition. A missing tooth may be associated with an extraction as a root cause, for example, due to a removed deciduous tooth or otherwise extracted tooth. A new tooth may be associated with an erupted tooth or a pontic tooth root cause. Calculating the probably of one or more root causes may also include identifying those discrepancies that have a high risk (e.g., high probability) of contributing to improper fit of the aligner. The risk associated with a discrepancy can depend on the type of discrepancy and the degree of the discrepancy. In one example, a discrepancy associated with a current tooth dentition model tooth shape that is “wider” than an original tooth dentition model tooth shape may be identified as being “high risk,” whereas a discrepancy associated with an original tooth dentition model tooth shape that is “wider” than a current tooth dentition model tooth shape may be identified as being “low risk.” See, e.g., FIG. 7.

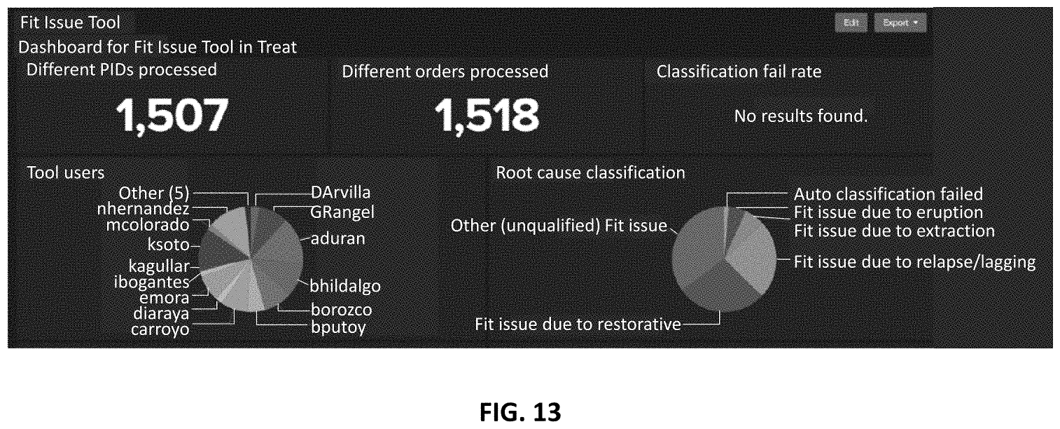

At 411, the probability of the root causes of the improper fitting aligner are displayed on the user interface. The details of the information displayed, and the display style may vary. In some examples, the root causes are graphically displayed (e.g., pie chart, bar graph and/or line chart). Other information, such as percentage of classification failed, may also be displayed. See, e.g., FIG. 13. The dental practitioner may use this information as a guide to investigate further. For example, the dental practitioner may decide how to modify the treatment plan based on whether the probably due to relapse/lagging is high, or whether the probably due to tooth eruption or extraction is high.

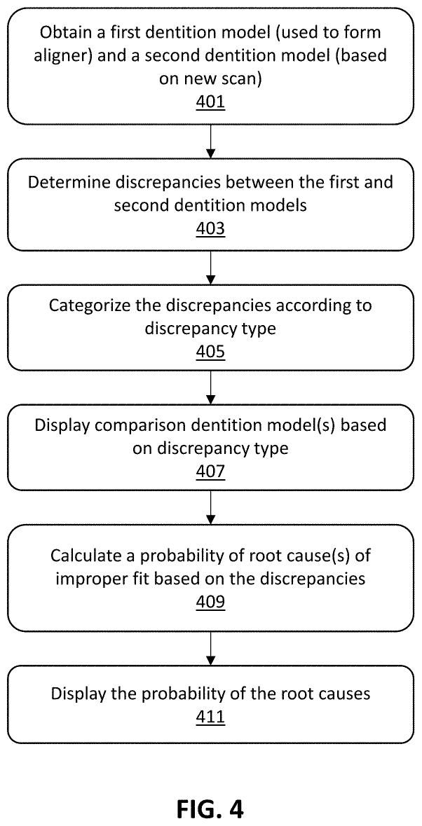

FIG. 5 illustrates a user interface showing an original dentition model 500 (e.g., first dentition model) that was used as a basis to form a dental aligner. The original dentition model 500 may be an interactive digital 3D representation where user may rotate, zoom in/out and click on particular features. In this case, the comparison dentition model 500 is accessible as a tab of the user interface. The original dentition model 500 may correspond to a target dentition for a stage in a dental treatment plan. In this example, the dentition model 500 includes a number of dental attachments, e.g., 505, which have been added to the dentition model 500 based on a chosen dental treatment plan. The user interface may also show tooth numbers, e.g., 507, of each tooth, which may be presented over or near a corresponding tooth in the user interface. In some examples, the dentition model 500 is presented within a tab of the user interface.

FIG. 6 illustrates a user interface showing a comparison dentition model 600, which is based on a comparison of an original dentition model (e.g., first dentition model) and a current dentition model of the patient's teeth (e.g., second dentition model). The current dentition model of the patient's teeth may be based on a new scan of the patient's teeth, and corresponds to a current configuration of the patient's dentition. The comparison dentition model 600 may be an interactive digital 3D representation where user may rotate, zoom in/out and click on particular features. In this case, the comparison dentition model 600 is accessible as a second tab in user interface, where the user may switch between viewing an original dentition model and the comparison dentition model 600. The comparison dentition model 600 may be derived by identifying discrepancies between the current dentition model and the original dentition model at different locations. In this example, the discrepancies are indicated with shaded regions. Specifically, a first color/shading 602 indicates a location on a tooth of the current dentition model (e.g., new scan) is wider than the same location of the tooth in the original dentition model, and a second color/shading 604 (e.g., different than the first color/shading) indicates a location on a tooth of the original dentition model 500 is wider than the same location of the tooth in the current dentition model (e.g., new scan). Greater intensity and/or darkness of the shading/color can be associated with a higher degree of discrepancy.

FIG. 7 illustrates another comparison dentition model 700, in this case, showing an aerial view of the incisor teeth. In this example, a second color/shading 704 indicates locations where incisor teeth of the original dentition model are wider than the same teeth in the current dentition model (e.g., new scan). These discrepancies be considered “low risk” for causing fit issues. In addition, a first color/shading 702 indicates locations where the teeth of the current dentition model (e.g., new scan) are wider than the same teeth in the original dentition model. These discrepancies be considered “high risk” for causing fit issues. Thus, in FIG. 7 the image shows an indicator of the probability that the dental aligner will improperly fit the patient's dentition (e.g., based on the color shading indicating regions of higher risk).

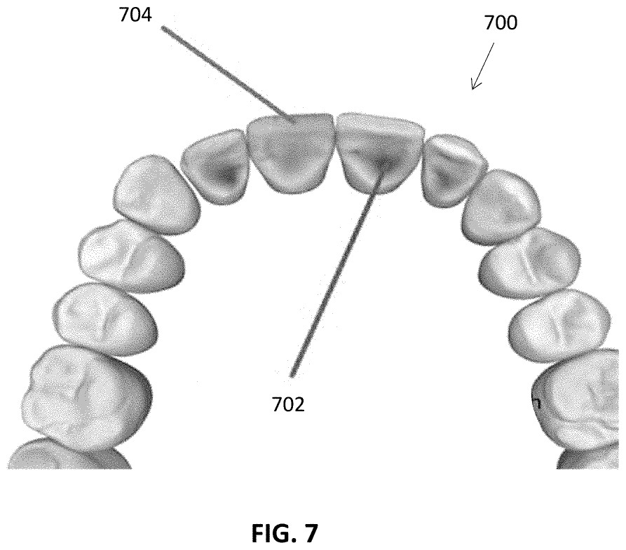

FIG. 8 illustrates an exemplary user interface display window showing a brief summary of shape discrepancy metrics between an original dentition model and a current dentition model. This provides numerical metrics of the teeth shape and positional differences.

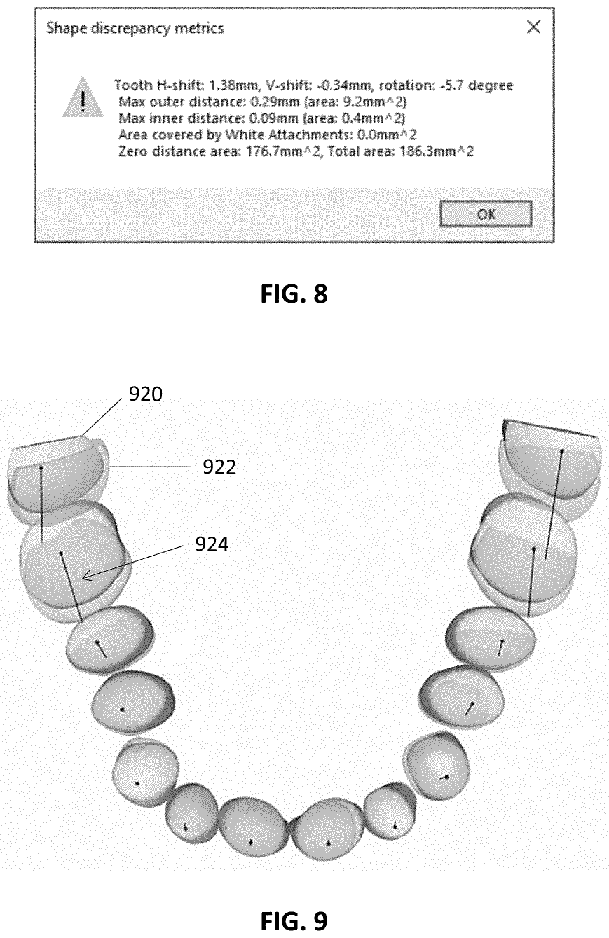

FIGS. 6 and 7 show comparison dentition models in a “heat map” visualization mode, in which shape discrepancies between an original dentition model and a current dentition model are highlighted. FIG. 9 illustrates an example a comparison dentition model displayed using a “tooth shifts” visualization mode, which highlights shifts in the individual teeth. FIG. 9 shows an aerial view of the comparison dentition model. In this case, the comparison dentition model includes a current dentition model 920 in a semitransparent view overlayed on an original dentition model 922, so that changes in the positions of the teeth can be seen. In addition, positional changes of each tooth from the original dentition model 922 and the current dentition model 920 are indicated with vectors, e.g., 924.

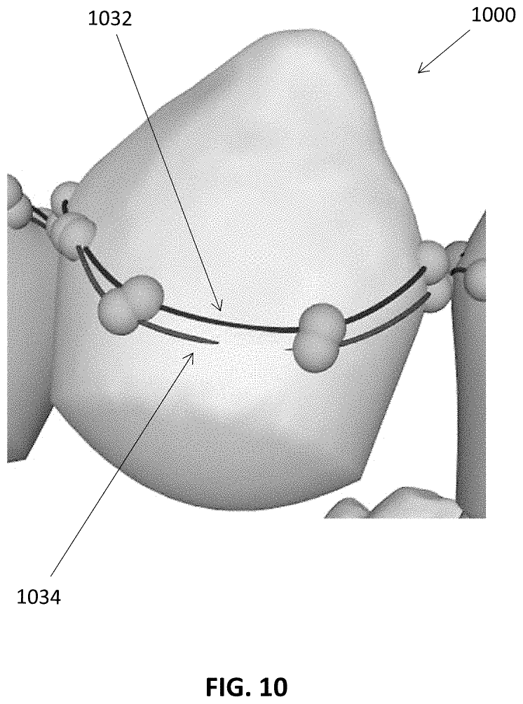

FIG. 10 illustrates an example of a comparison dentition model 1000 showing gingival lines 1032 and 1034 indicate the location of gingival lines associated with a particular tooth. In this case, a first gingival line 1032 indicates the location of the gingival line in the original dentition model, and a second gingival line 1034 indicates the location of the gingival line in the current dentition model. This view allows the user to observe positional changes that occurred. This information may be used to determine whether changes in the gum line may be a root cause of the improper fitting of the aligner. For example, certain gingival line changes may cause the aligner to rub the gingiva, potentially causes patient discomfort. Note that the spheres FIG. 10 indicate other aspects related to the gingival lines 1032 and 1034.

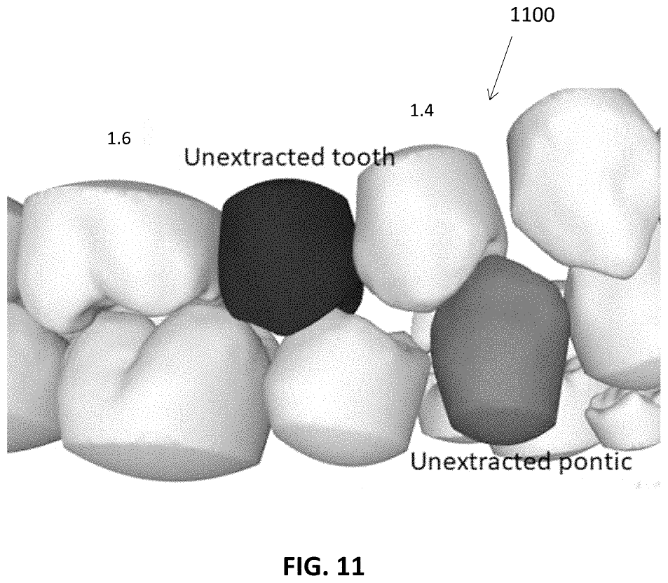

FIG. 11 illustrates an example of a comparison dentition model 1100 showing the location of an “unextracted tooth” and an “unextracted pontic” tooth. This view allows for visualization of mismatched extracted or unextracted teeth. The view may indicate that a tooth or pontic that was intended to be extracted was not (or not yet) extracted.

FIG. 12A illustrates an exemplary toolbar with controls for viewing aspects of one or more dentition models. The toolbar includes user accessible tabs 1202 related to various aspects of designing and choosing one or more dental treatment plans for a patient's dentition. FIG. 12A shows a “Fit Issues” tab selected, which includes controls for viewing a comparison dentition model. This user interface displays: a user interactive control to control a degree of transparency 1206; a user interactive control to control shape of the original (referred to as “complaint”) dentition model, current dentition model (referred to as “warranty jaw”), and a tooth of the original dentition model (referred to as a “warranty tooth”) 1208; a user interactive control to control whether to visualize (un)extracted teeth and teeth shifts 1210; a user interactive control to control whether to visualize gingival lines of original (“complaint LATs”) dentition model and the current (“warranty LATs”) dentition model 1212; a brief summary (“conclusion”) of root cause results of the fit issue analysis 1214; and a user interactive control to control different display scenarios 1216. In this example, the fit analysis tool provided a result (in the “conclusion” summary 1214) that the root cause of the improper fitting aligner relates to relapse/lagging of the treatment on the patient's dentition. FIG. 12B illustrates a close-up view of an exemplary drop-down menu for display scenarios 1216 in FIG. 12A. In the example of FIG. 12B, the user may choose to display the comparison dentition model in a “default visual mode,” an “extraction scenario,” a “restorative scenario,” an “eruption scenario,” or a “relapse scenario.”

FIG. 13 illustrates an example “Dashboard” view of the Fit Issue Tool that summarizes statistical findings of the fit issue analysis. Statistical data may include the tool users (e.g., names/identification of dental professionals) and the root cause classification, which may be displayed in a pie graph as shown in the example of FIG. 13. Examples of root cause classification may include fit issue due to tooth eruption, fit issue due to tooth extraction, fit issue due to relapse/lagging (i.e., patient's dentition is not on track with dental plan), fit issue due to a restorative procedure, and other (unqualified) fit issue. If a root cause is not able to be determined, the root cause classification may indicate the classification analysis failed. In the example of FIG. 13, the number of different patients (e.g., PIDs) processed, the number of different orders processed, and the classification fail rate are also displayed.

FIG. 14 is a diagram showing an example of a fit issue tool 1400. The fit issue tool 1400 may be incorporated into a portion of another system (e.g., a general treatment planning system) and may therefore also be referred to as a sub-system. In some cases, the fit issue tool 1400 may be an add-on (e.g., plug-in) to the general treatment planning system. In any of the method and apparatuses described herein the fit issue tool 1400 may be invoked by a user control, such as a tab, button, etc., as part of treatment planning system, or may be separately invoked. In FIG. 14, the fit issue tool 1400 may include a plurality of engines and datastores. The fit issue tool 1400 may include or be part of a computer-readable medium, and may include an input engine 1414 (e.g., providing and/or allowing access to the patient's original dentition model used to fabricate an aligner of interest, and the patient's current dentition model, such as obtained by a new scan). All or some of the original and current dentition model data may be stored in a datastore 1412.

The fit issue tool 1400 may include a discrepancy identifier engine 1402 that may compare the original dentition model and the current dentition model to determine points (e.g., in 3D meshes) where the models differ. The original and/or current dentition models may be formatted, scaled or normalized such that an accurate comparison may be made. The fit issue tool 1400 may also include a discrepancy categorizing engine 1404 that may categorize the identified discrepancies by discrepancy type. The discrepancy type may include, for example, tooth shape, tooth position, gingival line positions, presence of an extracted tooth, presence of an erupted tooth, or other types. The discrepancy type may include sub-types, such as increases/decreases in tooth shape, direction of tooth position change, gingival line recession or ascension, or other sub-types. The fit issue tool 1400 may also include an interactive display engine 1406 that displays an interactive user interface. The user interface may display the original dentition model, the current dentition model, and/or a comparison dentition model, which shows differences/discrepancies between the original and current dentition models. The user interface may include one or more tabs and/or tool bars that provide the user controls for viewing the discrepancies, for example, based on type. The fit issue tool 1400 may also include a root cause probability engine 1408 that associates the discrepancies with root causes, and calculates probabilities of the root causes. The root cause probability engine 1408 may determine risks associated with the various discrepancies, and identify those discrepancies that have a high risk of contributing to the improper fit of the dental aligner. An output engine 1410 may output results from the root cause analysis, for example, in the user interface.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for digital simulation of a retentiveness of a dental aligner, the method comprising:
initializing a 3D model of a patient's dentition based on a stage of a treatment plan corresponding to the dental aligner;
generating a plurality of zones, each having one or more teeth from the 3D model of the patient's dentition;
simulating a retention value for each of the zones, based on one or more angles between one or more surface normal vectors of one or more teeth within each zone, and one or more reference vectors parallel to a long axis of each of the one or more teeth within each zone; and
outputting one or more indicators of the probability that the dental aligner will improperly fit the patient's dentition based on the retention values.

2. The method of claim 1, further comprising outputting one or more attachment locations on the patient's dentition based on the retention values.

3. The method of claim 1, wherein the 3D model of the dentition corresponds to the detention at initiation of the stage of the treatment plan.

4. The method of claim 1, wherein the treatment plan includes multiple stages for moving teeth of the dentition toward a final position, wherein each of the multiple stages is associated with a corresponding aligner.

5. The method of claim 4, further comprising repeating the steps of initializing, generating, simulation and outputting for each of a plurality of aligners of the different stages of the treatment plan.

6. The method of claim 1, wherein generating the plurality of zones comprises:
dividing the 3D model into zones each having one or more teeth; and
wherein the retention value of each zone is based on an average retention value for the one or more teeth in the corresponding zone.

7. The method of claim 6, wherein simulating the retention value comprises:
determining a reference vector parallel to a long axis of a tooth;
determining the surface normal vectors distributed across a surface of the tooth and normal to the surface of the tooth; and
calculating angles between the reference vector and each of the surface normal vectors.

8. The method of claim 1, wherein simulating the retention value of the zones is based on a number of angles greater than 90 degrees.

9. The method of claim 1, further comprising determining a shape and size of one or more retention-enhancing attachments at the one or more attachment locations.

10. The method of claim 1, further comprising: adding one or more retention-enhancing attachments at the one or more attachment locations to the stage of the treatment plan; and modifying the treatment plan based on the addition of the one or more retention-enhancing attachments.

11. The method of claim 10, further comprising fabricating one or more dental aligners based on the modified treatment plan.

12. A non-transient, computer-readable medium containing program instructions for modifying a treatment plan based on one or more estimates of retentiveness of a dental aligner, the program instructions causing a processor to:
initialize a 3D model of a patient's dentition based on the stage of a treatment plan corresponding to the dental aligner;
generate a plurality of zones, each having one or more teeth from the 3D model of the patient's dentition;
simulate a retention value for each of the zones, based on one or more angles between one or more surface normal vectors of one or more teeth within each zone, and one or more reference vectors parallel to a long axis of each of the one or more teeth within each zone; and
output one or more indicators of the probability that the dental aligner will improperly fit the patient's dentition based on the retention values.

13. The non-transient computer-readable medium of claim 12, wherein the instructions further cause the processor to output one or more attachment locations on the patient's dentition based on the retention values.

14. The non-transient, computer-readable medium of claim 12, wherein the generating the plurality of zones wherein generating the plurality of zones comprises:
dividing the 3D model into zones each having one or more teeth; and
wherein the retention value of each zone is based on an average retention value for the one or more teeth in the corresponding zone.

15. The non-transient, computer-readable medium of claim 12, wherein the retention value for each of the zones is based on a number of angles greater than 90 degrees.

16. The non-transient, computer-readable medium of claim 12, wherein simulating the retention value comprises:
determining a reference vector parallel to a long axis of a tooth;
determining the surface normal vectors distributed across a surface of the tooth and normal to the surface of the tooth; and
calculating angles between the reference vector and each of the surface normal vectors.

17. The non-transient, computer-readable medium of claim 12, wherein simulating the retention value of the zones is based on a number of angles greater than 90 degrees.

18. The non-transient, computer-readable medium of claim 12, wherein the instructions further cause the processor to determine a shape and size of one or more retention-enhancing attachments at the one or more attachment locations.

19. The non-transient computer-readable medium of claim 12, wherein the instructions further cause the processor to add one or more retention-enhancing attachments at the one or more attachment locations to the stage of the treatment plan; and modify the treatment plan based on the addition of the one or more retention-enhancing attachments.

20. The non-transient, computer-readable medium of claim 12, wherein each surface normal vector is associated with a polygon of a surface mesh of the tooth, and wherein the simulated retention value is further based on a direction of each of the angles between the reference vector and corresponding surface normal vector.

21.-40. (canceled)
